(19) United States
(12) Patent Application Publication
(10) Pub. No.: US 2008/0031904 A1
(43) Pub. Date: Feb. 7, 2008

(54) Combination consisting of a magnesium preparation having a prolonged release and of another active substance, and use thereof in the fields of cosmetics, therapeutics and/or nutrition

(76) Inventor: Fabienne Menvielle-Bourg-Joanny, Paris (FR)

Correspondence Address: OSTROLENK FABER GERB & SOFFEN, 1180 AVENUE OF THE AMERICAS, NEW YORK, NY, 100368403, US

(21) Appl. No.: 11/286,192

(22) Filed: Nov. 23, 2005

Related U.S. Application Data
(63) Continuation-in-part of application No. PCT/FR04/01305, filed on May 26, 2004.

(30) Foreign Application Priority Data
May 27, 2003 (FR) 03.06411

(51) Int. Cl. A61K 8/97; A61K 8/18

(52) U.S. Cl. 424/401; 424/195.16; 424/195.17; 424/681; 424/725; 424/750; 424/757; 424/765

(57) ABSTRACT

The present invention relates to a combination useful in the fields of cosmetics, therapeutics and/or nutrition, for acting in particular against stress conditions, characterised in that it consists of (α) a sustained release preparation (I) comprising, in association with a physiologically acceptable excipient, a mixture of: (A) magnesium derived from a magnesium source consisting of MgO, MgCl₂ and hydrates of the formula MgCl₂·nH₂O, where n is a whole or fractional number greater than 0 or equal to 6, (B) at least one substance selected from among (B1) a hydrophilic polymer, which is a cellulose derivative, and/or (B2) a hydrophobic substance belonging to the family of fatty acid esters and the mixtures thereof, and (C) an inert filler acting as diluent, in particular lactose; and (β) an active substance (Z), which is in particular selected from among plant extracts, yeast extracts, algae extracts, hormones, proteins, peptides, amino acids, unsaturated fatty acids and mixtures thereof.

### FIELD OF THE INVENTION

The present invention relates to a novel combination making use of a sustained release Mg-based preparation (I) (i.e. “delayed release Mg”), on the one hand, and at least one active substance (Z) defined below, on the other hand, said combination being useful in the fields of cosmetics, therapeutics and/or nutrition. This novel combination is of particular relevance in cosmetics and dermopharmacy vis-à-vis stress conditions, and in the field of nutrition as a nutritional supplement. This combination of I+Z may be formulated either in a single dosage form, or in distinct or separate dosage forms in the context of combined medicinal, nutritional and/or cosmetic treatment.

### PRIOR ART

The closest prior art consists of granted European Patent EP 0542979 B. The object of said patent is “a therapeutic composition useful vis-à-vis magnesium deficiencies and intended to ensure that the magnesium which it contains is released slowly and continuously in the form of assimilable Mg²⁺ in the intestine, so as to make up the daily intake of magnesium in man to at least 6 mg/kg, said composition being characterised in that it contains a mixture comprising, in association with a physiologically acceptable excipient,

(a) 4 to 14 parts by weight of magnesium derived from a magnesium source consisting of MgO, MgCl₂ and magnesium chloride hydrates of the formula MgCl₂·n(H₂O), where n is a whole or fractional number greater than 0 and less than or equal to 6,

(b) 6 to 13 parts by weight of a hydrophilic polymer, when the Mg source is MgO, or 10 to 30 parts by weight of a hydrophobic substance selected from the group consisting of physiologically acceptable hydrophobic polymers, fatty acid esters and mixtures thereof, when the Mg source is other than MgO, and

(c) 6 to 16 parts by weight of an inert filler present as a solid diluent and selected especially from the group consisting of lactose, alkali metal and alkaline earth metal phosphates and mixtures thereof,

the Mg content of said mixture being between 5 and 60% by weight based on the weight of said mixture.”

The technical solution provided by patent EP 0542979 B is admittedly very effective. However, it has been noted that (i) oral use in one and the same core of a hydrophobic polymer (such as PVC) and MgCl₂·n(H₂O) and (ii) that of a hydrophilic polymer (such as PVP) and MgO cause difficulties with regard to the regulations of certain member states of the European Union.

To overcome these difficulties, it is proposed to provide a new technical solution which is distinguished from the teaching of the prior patent by elimination of the PVC and PVP, on the one hand, and in particular by combination of the sustained release Mg composition with at least one other active substance (Z), on the other hand.

It has in fact surprisingly been found that such a combination exhibits a synergistic effect relative to the magnesium [provided in the aforesaid form of MgO, MgCl₂ or MgCl₂·n(H₂O)] and to Z, when the preparation I and the substance Z are administered separately or in a single dosage form.

### SUBJECT MATTER OF THE INVENTION

According to a first aspect of the invention, a novel combination is provided vis-à-vis magnesium deficiencies, which is useful in particular

(i) in the field of therapeutics, as a medicament,

(ii) in the field of cosmetics, and/or

(iii) in the field of nutrition, as a nutritional supplement.

More precisely, the invention advocates a novel combination useful in the fields of cosmetics, therapeutics and/or nutrition, for acting in particular against stress conditions, characterised in that it consists of

(α) a sustained release magnesium preparation (I) comprising, in association with a physiologically acceptable excipient, a mixture of:
(A) magnesium derived from a magnesium source consisting of MgO, MgCl₂ and hydrates of the formula MgCl₂·nH₂O, where n is a whole or fractional number greater than 0 or equal to 6,
(B) at least one substance selected from among B1, B2 or a combination thereof:
   (B1) a hydrophilic polymer, which is a cellulose derivative, and/or
   (B2) a hydrophobic substance belonging to the family of fatty acid esters with polyols, and
(C) an inert filler acting as diluent, in particular lactose, the magnesium content being between 1 and 60% by weight relative to the weight of said mixture A+B+C; and

(β) an active substance (Z), which is selected from the group consisting of:
natural plant extracts,
yeasts and yeast extracts,
algae extracts,
hormones,
proteins, in particular enzymes,
peptides,
amino acids,
unsaturated fatty acids, and
mixtures thereof.

The invention also advocates use of the present combination to supplement daily magnesium intake, in the cosmetic field, the dermatotherapeutic field and/or nutritional field.

Use of the preparation (I) and of said substance (Z) is also advocated to obtain a medicament intended for the treatment of magnesium deficiencies.

### DETAILED DESCRIPTION OF THE INVENTION

Advantageously, said mixture of A, B and C forms a sustained release core which is capable of being accommodated inside a gastroresistant coating, of the film coating type.

The hydrophilic polymer B1 is advantageously selected from among cellulosic polymers derived from cellulose: in particular carboxymethylcellulose, hydroxypropylcellulose, hydroxypropylmethylcellulose and mixtures thereof.

The hydrophobic substance B2 is advantageously a fatty acid ester, where said fatty acid is a C₈-C₂₄ fatty acid and the alcohol component residue comprises at least one polyol residue (such as glycerol and/or polyethylene glycol). Said substance B2 may be a mixture of fatty acid esters. In particular, the hydrophobic substance may be glycerol palmitate and/or glycerol behenate.

The inert filler (C) is advantageously lactose (preferably anhydrous lactose).

The mixture of A+B+C may contain other additives conventional in pharmaceutical formulation, in particular silica (colloidal silica) and/or a lubricant. The magnesium content of said mixture of A+B+C will be between 1 and 60% by weight.

As indicated above, the gastroresistant coating is advantageously a film coating. It is more particularly formed of acetylated monoglycerides and shellac or alternatively of cellulose acetophthalate, a cellulose acetophthalate/polyethylene glycol mixture, a cellulose acetophthalate/C₁-C₅ alkyl phthalate mixture or a cellulose acetophthalate/polyethylene glycol/C₁-C₅ alkyl phthalate mixture.

In practice, in said preparation (I):

the weight ratio of B:A will be between 0.8:1 and 8.2:1 (preferably between 1.2:1 and 4.8:1),

the weight ratio of C:A will be between 0.4:1 and 4:1 (preferably between 0.5:1 and 2.3:1), and

the quantity of Mg per dosage unit, in particular in the form of a tablet, will vary between 10 mg/dosage unit and 250 mg/dosage unit, the preferred dosages of Mg being 30 mg/dosage unit, 50 mg/dosage unit or 100 mg/dosage unit.

Advantageously, the Mg content in said preparation I will be between 1 and 60% by weight relative to the weight of said preparation I.

The preparation I and each formulation containing Z may be administered separately (i.e. in distinct dosage forms) in the context of combined therapy or combined treatment, in particular internally (perorally) for both or alternatively internally (perorally) for I and externally (topically) for a composition containing Z. As a variant, it is possible to provide peroral administration of a single product consisting of I+Z (i.e. a single dosage form), the active substance(s) Z then being incorporated:

(i) in the core containing A, B and C,

(ii) in the shell provided by the gastroresistant coating of said preparation (I),

(iii) in a non-gastroresistant layer surrounding the core containing A, B and C, or

(iv) in a layer surrounding said gastroresistant coating.

In the case of administration in a single dosage form, the substance Z may be added to the magnesium source or is capable of replacing a portion of the magnesium provided by said source.

It is beneficial for there to be a non-gastroresistant layer around the preparation I or around the gastroresistant shell if it is necessary for a substance Z contained therein to be released quickly.

Examples of suitable substances Z worthy of particular mention are plant extracts, for example hawthorn extract, valerian extract, balm extract, sea thyme extract, maritime pine bark extract, lime tree sapwood extract, cereal extracts (in particular wheat and/or rice protein hydrolysate), soya extract or a mixture thereof. Other substances Z which may be mentioned are fruit extracts (in particular apple extract, melon extract, papaya extract, pineapple extract), yeast, yeast extract, algae extract or a mixture thereof.

The substance Z may also be selected from the group consisting of hormones, proteins, peptides, amino acids, or a mixture thereof.

From a practical point of view, the plant extracts used are those known as medicinal, which are prepared in accordance with the pharmacopoeia. For the cereal (such as wheat and rice) and soya extracts, it is possible, according to the invention, to use vegetable protein hydrolysates; for example wheat gluten hydrolysate or rice protein hydrolysate, which each contain peptides and amino acids.

Fruit extracts are also recommended, for example optionally fermented papaya extract, medicinal pineapple extract, which is rich in bromelain, and melon extract, which is rich in superoxide dismutase (SOD).

The unsaturated fatty acids useful according to the invention are C₁₂-C₂₄ unsaturated fatty acids. Particularly suitable are oleic acid, linoleic acid, linolenic acid, an ω³ acid, an ω⁶ acid or a mixture thereof. More particularly recommended is apple extract, which is rich in ω³.

Other substances Z which may be added are one or more vitamins (in particular vitamin B12, vitamin E and/or vitamin D) and one or more trace elements or minerals (in particular Zn, Mn, Cu).

In the field of cosmetics, dermopharmaceuticals and nutrition,

(i) vis-à-vis stress and ageing affecting the dermis and the epidermis, it is recommended to combine preparation I and balm extract, in a single oral dosage form,

(ii) vis-à-vis disorders associated with the menopause, it is recommended to combine preparation I and a soya extract containing daidzin and/or genistin, and vis-à-vis oxidising agents a maritime pine extract rich in proanthocyanidols, in a single oral dosage form or alternatively in the form of two different dosage forms, and

(iii) vis-à-vis skin care, it is recommended, for example, to administer preparation I orally and essence of lavender topically (in particular in a bath).

Other advantages and features will be better understood on reading the following exemplary embodiments, which are in no way limiting.

### EXAMPLE 1

### Sustained Release Tablets containing a 100 mg Dose of Magnesium

Film-coated tablets were prepared in accordance with the method of Example 1 of EP 0542979 B, replacing MgO with MgCl₂·6H₂O, each tablet having

a core containing:

| | | |
|---|---|---|
| MgCl₂•6H₂O | 836.45 mg | |
| anhydrous lactose (diluent) | 52.64 mg | C:A = 0.52:1 |
| hydrated colloidal silica (binder) | 13.03 mg | |
| hydroxypropylmethylcellulose | 120.00 mg | |
| (gelling agent) | | |
| glycerol palmitate (lubricant) | 112.88 mg | B:A = 2.32:1 |

and a film coating.

### EXAMPLE 2

### Sustained Release Tablets containing a 50 mg Dose of Magnesium

Film-coated tablets were prepared as indicated above, each tablet having a core containing:

| | | |
|---|---|---|
| MgCl₂•6H₂O | 418.22 mg | |
| anhydrous lactose (diluent) | 27.38 mg | C:A = 0.54:1 |
| hydrated colloidal silica (binder) | 10.00 mg | |
| hydroxypropylmethylcellulose | 55.00 mg | |
| (gelling agent) | | |
| glycerol palmitate (lubricant) | 61.40 mg | B:A = 2.33/1 |

and a gastroresistant coating.

### EXAMPLE 3

### Sustained Release Tablets containing a 30 mg Dose of Magnesium and 40 mg of Hawthorn

Film-coated tablets were prepared as indicated above, each tablet having a core containing:

| | | |
|---|---|---|
| MgCl₂•6H₂O | 250.93 mg | |
| medicinal hawthorn extract | 40.00 mg | |
| anhydrous lactose (diluent) | 27.38 mg | C:A = 0.91/1 |
| hydrated colloidal silica (binder) | 10.00 mg | |
| hydroxypropylmethylcellulose | 55.00 mg | |
| (gelling agent) | | |
| glycerol palmitostearate (lubricant) | 61.49 mg | B:A = 3.88:1 |

and a film coating.

### EXAMPLE 4

### Sustained Release Tablets containing a 50 mg Dose of Magnesium and 50 mg of Hawthorn

Film-coated tablets were prepared as indicated above, each tablet having a core containing:

| | | |
|---|---|---|
| MgCl₂•6H₂O | 418.22 mg | |
| medicinal hawthorn extract | 50.00 mg | |
| anhydrous lactose (diluent) | 52.64 mg | C:A = 1.10:1 |
| hydrated colloidal silica (binder) | 13.03 mg | |
| hydroxypropylmethylcellulose | 120.00 mg | |
| (gelling agent) | | |
| glycerol palmitate (lubricant) | 112.88 mg | B:A = 4.65/1 |

and a gastroresistant coating.

### EXAMPLE 5

### Sustained Release Tablets containing a 100 mg Dose of Magnesium plus Wheat Protein Hydrolysate

Tablets were prepared as indicated above, each tablet having a core containing:

| | |
|---|---|
| MgO | 165.84 mg |
| Wheat gluten hydrolysate | 116.16 mg |
| (sold under the name | |
| HYPROBLE ® and containing 27 to 32% | |
| by weight of peptides/proteins and less | |
| than 5% by weight of free amino acids) | |
| anhydrous lactose | 50.00 mg |
| colloidal silica | 13.03 mg |
| hydroxypropylmethylcellulose | 110.00 mg |
| glycerol mono- and distearate | 80.77 mg |
| magnesium stearate | 12.20 mg |

and a gastroresistant coating

based on shellac.

### EXAMPLE 6

### Sustained Release Tablets containing a 100 mg Dose of Magnesium plus other Ingredients

Tablets were prepared as indicated above, each tablet having a core containing:

| | |
|---|---|
| HYPROMAG ® | 286 mg |
| (mixture of marine MgO, supplying | |
| 100 mg of Mg, and 118.16 mg of rice | |
| protein hydrolysate) | |
| medicinal hawthorn extract | 30 mg |
| medicinal Californian poppy extract | 10 mg |
| anhydrous lactose | 50.00 mg |
| colloidal silica | 13.03 mg |
| hydroxypropylmethylcellulose | 110.00 mg |
| glycerol mono- and distearate | 80.77 mg |
| magnesium stearate | 12.20 mg |

and a gastroresistant coating

based on shellac.

A final product is obtained which is particularly effective against stress and slight to moderate anxiety.

### EXAMPLE 7

### Combination of a Sustained Release Tablet containing Magnesium, and a Calendula Extract.

On the one hand, a tablet is prepared according to Example 1 above and, on the other hand, a topical preparation containing a medicinal Calendula extract.

This combination is intended for the treatment of skin stress orally (the tablet) and topically (the Calendula extract).

### EXAMPLE 8

### Combination of a Sustained Release Tablet containing Magnesium, and an Essential Oil of Lavender

On the one hand, a tablet is prepared according to Example 5 above and, on the other hand, a topical preparation containing an essential oil of lavender.

This combination is intended for the treatment of skin stress orally (the tablet) and topically (the lime tree sapwood extract).

### EXAMPLE 9

A tablet is prepared according to Example 5 above, without its gastroresistant coating. A polymeric layer (of the polyacrylate/polymethacrylate type) containing a maritime pine bark extract is deposited on the surface of said tablet. After drying, it is possible, if need be, to coat the resultant inner product with a gastroresistant coating.

### Dissolution Tests

The compositions according to the invention have a satisfactory dissolution profile, determined using an in vitro dissolution test performed under the following conditions:

Dissolution kinetics in vitro over 8 hours, in a buffer medium of pH 6.8, with sampling at 1 hour, 2 hours, 4 hours and 8 hours (starting with tablets which have spent 2 hours in a 0.1 N hydrochloric acid medium); and

determination of the magnesium by atomic absorption spectroscopy.

By way of non-limiting example, a dissolution test may be performed in the following manner:

A SOTAX AT7®, paddle apparatus is used, at a temperature of 37±0.5° C. A pH=6.8 buffer solution is prepared from 17 g of monopotassium phosphate in 5000 ml of water, adjustment to pH=6.8 being achieved by means of a 1 N sodium hydroxide solution. The dissolution volume is 1000 ml of pH=6.8 buffer in each reactor. The speed of rotation of the paddles is set at 100 rpm.

The test was performed using a tablet as described above comprising a gastroresistant coating.

Determination of the magnesium by atomic absorption is performed on a 1 ml sample. The 1 ml sample is diluted to 100 ml in water to which has been added 5 ml of a 100 g/l solution of strontium chloride. The dissolution profile curve is given in the appendix (FIG. 1/1).

A first phase is noted up to the end of 2 hours, in which a small quantity of magnesium is released; this is followed by a second phase from 2 to 4 hours, in which the greater part of the magnesium is released; and finally a third phase between 4 and 8 hours, in which the remainder of the magnesium is released.

The dissolution profile of the tablet studied demonstrates that the magnesium is released gradually and proves how effective the formulation is in meeting magnesium needs for a duration of greater than 8 hours.

In another dissolution test, the dissolved magnesium is also determined by atomic absorption spectrophotometry. A UNICAM® PU 9200 X flame spectrophotometer is used. The solution to be tested is diluted 100 times with B.

### Braun Inflac Aqua.

Samples are taken from the solution to be tested, respectively of 2.5 ml, 5 ml and 10 ml, and the volume is adjusted to 100 ml by means of a 0.2% caesium chloride solution in 0.5 M nitric acid. 25 ml samples of dissolution medium are taken after 1 hour and 2 hours, then diluted to 50 ml using Inflac aqua. The solutions are filtered and 5 ml of each filtrate are diluted to 100 ml with a 0.2% caesium chloride solution in 0.5 M nitric acid.

Other 25 ml samples of dissolution medium are taken after 4 hours and 8 hours, then diluted to 50 ml using Inflac aqua. The solutions are filtered and 1 ml of each filtrate is diluted to 100 ml with a 0.2% caesium chloride solution in 0.5 M nitric acid. The magnesium concentration of each of the 4 solutions is determined.

It may be noted that, after 4 hours, 80% of the magnesium present has been released and that, after 8 hours, 95% of the magnesium has been released.

The above-stated advantages are also confirmed by this test.

The compositions of Examples 2, 3 and 4 give similar results in the dissolution tests described above.

### Antiradical Resistance Tests

Experiments measuring resistance to the action of free radicals generated in situ from a free radical generator were carried out (i) on rat erythrocytes receiving the tablets or combinations according to the invention, and (ii) on human corneocyte cultures which have previously been brought into contact with the diluted constituents of the tablets and combinations according to the invention, following the operating methods described in European Patent EP 0418335 B.

Resistance to stress is determined by measuring the free resistance expressed as the 50% lysis time (T_{1/2}) of the cells subjected to the action of the free radical field. The results obtained demonstrate the synergistic effect of the technical solution provided by the invention vis-à-vis the sum (obtained by calculation) of the effects of Mg administered alone and the substance Z administered alone.

1. A combination useful in the fields of cosmetics, therapeutics and/or nutrition, for acting in particular against stress conditions, characterised in that it consists of
(α) a sustained release magnesium preparation (I) comprising, in association with a physiologically acceptable excipient, a mixture of:
(A) magnesium derived from a magnesium source consisting of MgO, MgCl₂ and hydrates of the formula MgCl₂·nH₂O, where n is a whole or fractional number greater than 0 or equal to 6,
(B) at least one substance selected from among B1, B2 or a combination thereof:
(B1) a hydrophilic polymer, which is a cellulose derivative, and/or
(B2) a hydrophobic substance belonging to the family of fatty acid esters with polyols, and
(C) an inert filler acting as diluent, in particular lactose, the magnesium content being between 1 and 60% by weight relative to the weight of said mixture A+B+C; and,
(β) an active substance (Z), which is selected from the group consisting of:
natural plant extracts,
yeasts and yeast extracts,
algae extracts,
hormones,
proteins, in particular enzymes,
peptides,
amino acids,
unsaturated fatty acids, and
mixtures thereof.

2. A combination according to claim 1, wherein said mixture of A, B and C forms a sustained release core which is capable of being accommodated inside a gastroresistant coating, of the film coating type.

3. A magnesium-based combination according to claim 1, wherein said active substance (Z) is capable of replacing a portion of the magnesium contained in said preparation (I).

4. A combination according to claim 1, wherein said preparation (I) and said active substance (Z) comprise distinct dosage forms for separate administration.

5. A combination according to claim 1, wherein said preparation (1) and said active substance (Z) are in the same dosage form.

6. A combination according to claim 4, wherein (i) said preparation (I) is in the form of an oral dosage form comprising a core containing A, B and C and which is capable of being accommodated inside a gastroresistant coating, and in that (ii) said active substance (Z) is in the form of a topical dosage form.

7. A combination according to claim 5, wherein said preparation (I) and said active substance (Z) are in the same dosage form, said active substance (Z) being incorporated
(i) in the core containing A, B and C,
(ii) in the shell provided by the gastroresistant coating of said preparation (I),
(iii) in a non-gastroresistant layer surrounding the core containing A, B and C, or
(iv) in a layer surrounding said gastroresistant coating.

8. A combination according to claim 1, wherein in said preparation (I):
the weight ratio of B:A is between 0.8:1 and 8.2:1 (preferably between 1.2:1 and 4.8:1),
the weight ratio of C:A is between 0.4:1 and 4:1 (preferably between 0.5:1 and 2.3:1), and
the quantity of Mg per dosage unit, in particular in the form of a tablet, varies between 10 mg/dosage unit and 250 mg/dosage unit, the preferred dosages of Mg being 30 mg/dosage unit, 50 mg/dosage unit or 100 mg/dosage unit.

9. A combination according to claim 1, wherein said substance (Z) is selected from the group consisting of hawthorn extract, valerian extract, balm extract, sea thyme extract, maritime pine bark extract, lime tree sapwood extract, cereal extracts (in particular wheat and/or rice protein hydrolysate), soya extract or a mixture thereof.

10. A combination according to 1, wherein said substance (Z) is selected from the group consisting of fruit extracts (in particular apple extract, melon extract, papaya extract, pineapple extract), yeast extract, algae extract or a mixture thereof.

11. A combination according to claim 1, wherein said substance (Z) is selected from the group consisting of hormones, proteins, peptides, amino acids, or a mixture thereof.

12. A combination according to claim 1, wherein said substance (Z) is selected from the group consisting of C₁₂-C₂₄ unsaturated fatty acids, in particular oleic acid, linoleic acid, linolenic acid, an ω³ acid, an ω⁶ acid or a mixture thereof.

13. Use of the combination according to claim 1, wherein in the field of cosmetics, in the field of dermatotherapeutics and/or in the field of nutrition.
